(19) United States
(12) Patent Application Publication
(10) Pub. No.: US 2025/0206692 A1
(43) Pub. Date: Jun. 26, 2025

(54) HYDROGENATION OF CANNABIGEROL AND CANNABICHROMENE

(71) Applicant: Colorado Chromatography, LLC, Hudson, CO (US)

(72) Inventors: Arianna C. COLLINS, Hudson, CO (US); Kyle P. RAY, Hudson, CO (US); Westley CRUCES, Hudson, CO (US)

(73) Assignee: Colorado Chromatography, LLC, Hudson, CO (US)

(21) Appl. No.: 18/845,802

(22) PCT Filed: Jul. 22, 2022
(86) PCT No.: PCT/US2022/074054

§ 371 Date: Sep. 10, 2024

Related U.S. Application Data
(60) Provisional application No. 63/319,670, filed on Mar. 14, 2022.

(51) Int. Cl. C07C 37/00; B01J 23/44; C07D 311/74

(52) U.S. Cl. CPC C07C 37/003; B01J 23/44; C07D 311/74

(57) ABSTRACT

Methods for producing hydrogenated cannabigerol and cannabichromene derivatives are disclosed. The methods preserve the aromatic olefins while selectively reducing the nonaromatic olefins using hydrogen gas, a hydrogen gas source, or a mixture thereof and a catalyst to produce the corresponding hydrogenated derivatives, in some aspects, the source of hydrogen gas comprises ammonium formate and/or formic acid. In some aspects, the catalyst is Pd/C or Pt/C.

### BACKGROUND OF THE INVENTION

### I. Field of the Invention

This invention relates to the fields of organic chemistry and medicinal chemistry.

### II. Background

Cannabichromene (CBC) and cannabigerol (CBG) have low affinity for human CB1 and CB2 receptors. Increased levels of saturation, defined by the fraction of sp³ hybridized carbon atoms (Fsp³), makes compounds more three-dimensional in shape and increases the number of chiral centers, thereby increasing the number of isomers of each compound. Such effects on molecular shapes of CBC and CBG derivatives might allow for improved interactions with CB1 and CB2 receptors.

CBC, CBG, and related derivatives that include relatively planar olefin groups can serve as the bases for producing more complex derivatives having higher Fsp³. By employing CBC and CBG variants where the aryl-linked pentyl group has been changed, a number of CBC and CBG-related, higher Fsp³ derivatives can be produced. The new CBC and CBG derivatives can improve the success of drug development programs by providing architecturally-complex drug molecule candidates.

### SUMMARY OF THE INVENTION

The present provides methods for the synthesis of CBC and CBG derivatives with increased sp³ fraction. In some aspects, the CBC and CBG derivatives include an aromatic ring moiety, but are devoid of non-aromatic olefin groups. Some aspects of the disclosure are directed to a process for the preparation of a hydrogenated cannabigerol derivative, comprising providing a cannabigerol derivative of formula I to a reaction vessel:

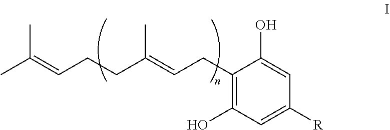

wherein n is an integer of from 0 to 4 and r a substituted or unsubstituted alkyl, alkenyl, or aryl group having from 1 to 10 carbon atoms, providing a catalyst to the reaction vessel, providing hydrogen gas, a source of hydrogen gas, or a combination thereof to the reaction vessel, and producing a hydrogenated cannabigerol derivative of formula II:

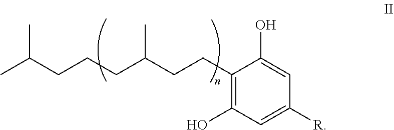

Some aspects of the disclosure are directed to a process for the preparation of a hydrogenated cannabichromene derivative, comprising providing a cannabichromene derivative of formula III to a reaction vessel:

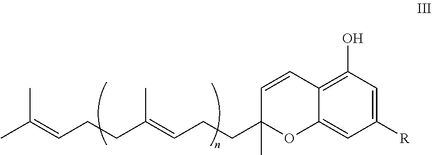

wherein n is an integer of from 0 to 4 and R is hydrogen or a substituted or unsubstituted alkyl, alkenyl, or aryl group having from 1 to 10 carbon atoms, providing a catalyst to the reaction vessel, providing hydrogen gas, a source of hydrogen gas, or a combination thereof to the reaction vessel, and producing a hydrogenated cannabichromene derivative of formula IV:

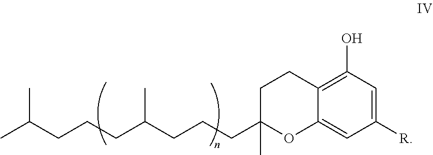

In some aspects, R is methyl, ethyl, propyl, butyl, pentyl, hexyl, heptyl, dimethylheptyl, octyl, phenyl, benzyl, isoprenyl, or geranylfarnesyl. In further aspects, R is a carbocycle or a heterocycle. In some aspects, R is CF₃, —CH₂F, —(CH₂)₂F, —(CH₂)₃F, —(CH₂)₄F, —(CH₂)₅F, —(CH₂)₆F, —(CH₂)₇F, or —(CH₂)₈F. In some aspects, the catalyst is provided in an amount ranging from 0.01 to 10 molar equivalents. The catalyst can be provided at any one of, less than, greater than, between, or any range thereof of 0.01, 0.02, 0.03, 0.04, 0.05, 0.06, 0.07, 0.08, 0.09, 0.10, 0.20, 0.30, 0.40, 0.50, 0.60, 0.70, 0.80, 0.90, 1.0, 1.1, 1.2, 1.3, 1.4, 1.5, 1.6, 1.7, 1.8, 1.9, 2.0, 2.1, 2.2, 2.3, 2.4, 2.5, 2.6, 2.7, 2.8, 2.9, 3.0, 3.1, 3.2, 3.3, 3.4, 3.5, 3.6, 3.7, 3.8, 3.9, 4.0, 4.1, 4.2, 4.3, 4.4, 4.5, 4.6, 4.7, 4.8, 4.9, 5.0, 5.1, 5.2, 5.3, 5.4, 5.5, 5.6, 5.7, 5.8, 5.9, 6.0, 6.0, 6.1, 6.2, 6.3, 6.4, 6.5, 6.6, 6.7, 6.8, 6.9, 7.0, 7.1, 7.2, 7.3, 7.4, 7.5, 7.6, 7.7, 7.8, 7.9, 8.0, 8.1, 8.2, 8.3, 8.4, 8.5, 8.6, 8.7, 8.8, 8.9, 9.0, 9.1, 9.2, 9.3, 9.4, 9.5, 9.6, 9.7, 9.8, 9.9, or 10.0 molar equivalents, such as such as 0.02 molar equivalents to 0.20 molar equivalents. In some aspects, the catalyst is selected from the group consisting of Pd/C, Pt/C, Rh/C, Ru/C, Raney nickel, Pd/alumina, Pd/activated charcoal, Pt/alumina, Pt/activated charcoal, Pt₂O (Adam's catalyst), Wilkinson's catalyst ([RhCl(PPh₃)₃]), Crabtree's catalyst ([CsH₁₂IrP(C₆Hₙ)₃CsHsN]PF₆), 9-borabicyclo[3.3.1]nonane, (R/S)-alpine borane, BH₃-DMSO, BH₃-THF, and N-methylimidodiacetic (MIDA) boronates. In some aspects, the catalyst is Pd/C or Pt/C. In some aspects, the hydrogen gas is provided in an amount that affords an intra-vessel gas pressure ranging from 1 bar to 20 bar. The hydrogen gas can be provided at any one of, less than, greater than, between, or any range thereof of 1, 2, 3, 4, 5, 6, 7, 8, 9, 10, 11, 12, 13, 14, 15, 16, 17, 18, 19, or 20 bar, such as 5 bar to 10 bar. In some aspects, the source of hydrogen gas generates hydrogen gas in situ. In some aspects, hydrogen gas is not provided to the reaction vessel. In some aspects, the source of hydrogen gas comprises ammonium formate and/or formic acid. In some aspects, the amount of ammonium formate ranges from 1 to 40 molar equivalents. In some aspects, the amount of formic acid ranges from 1 to 40 molar equivalents.

In some aspects, the process further comprises providing a solvent to the reaction vessel prior to the heating step. In some aspects, the solvent is selected from the group consisting of methanol, ethanol, propanol, isopropanol, butanol, tert-butanol, THF, 2-Me-THF, toluene, and ethyl acetate. In some aspects, the process further comprises the step of heating the reaction vessel. The reaction vessel may be heated to a temperature ranging from 25° C. to 100° C. The temperature can be any one of, less than, greater than, between, or any range thereof of 25, 26, 27, 28, 29, 30, 31, 32, 33, 34, 35, 36, 37, 38, 39, 40, 41, 42, 43, 44, 45, 46, 47, 48, 49, 50, 51, 52, 53, 54, 55, 56, 57, 58, 59, 60, 61, 62, 63, 64, 65, 66, 67, 68, 69, 70, 71, 72, 73, 74, 75, 76, 77, 78, 79, 80, 81, 82, 83, 84, 85, 86, 87, 88, 89, 90, 91, 92, 93, 94, 95, 96, 97, 98, 99, or 100° C., such as 50° C. to 60° C. In some aspects, the reaction vessel is hermetically sealed while the reaction is occurring. In some aspects, the process further comprises purging the reaction vessel with an inert gas prior to addition of reactants and catalyst. In some aspects, the inert gas is nitrogen or argon.

Some aspects of the disclosure are directed to a process for the preparation of tetrahydrocannabigerol (H4CBG) comprising providing cannabigerol (CBG) to a reaction vessel, providing a catalyst to the reaction vessel, providing hydrogen gas, a source of hydrogen gas, or a combination thereof to the reaction vessel, and producing H4CBG.

Some aspects of the disclosure are directed to a process for the preparation of tetrahydrocannabichromene (H4CBC), comprising providing cannabichromene (CBC) to a reaction vessel, providing a catalyst to the reaction vessel, providing hydrogen gas, a source of hydrogen gas, or a combination thereof to the reaction vessel, and producing H4CBC.

Some aspects of the disclosure are directed to a composition comprising a compound of formula II:

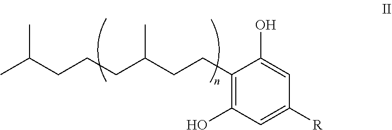

wherein n is an integer of from 0 to 4 and R is hydrogen or a substituted or unsubstituted alkyl, alkenyl, or aryl group having from 1 to 10 carbon atoms.

In some aspects, the compound of formula II is further defined as one of:

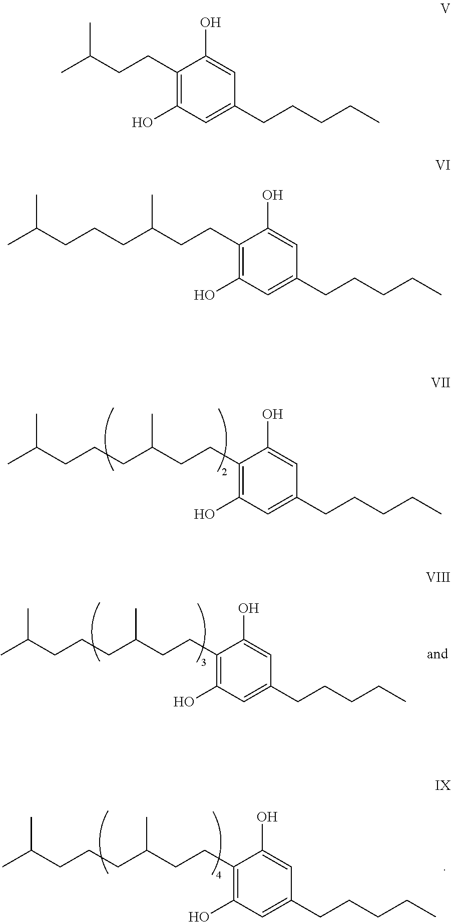

Some aspects of the disclosure are directed to a composition comprising a compound of formula IV:

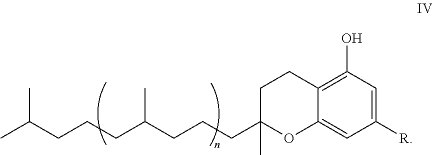

wherein n is an integer of from 0 to 4 and R is hydrogen or a substituted or unsubstituted alkyl, alkenyl, or aryl group having from 1 to 10 carbon atoms. In some aspects, the compound of formula IV is further defined as one of:

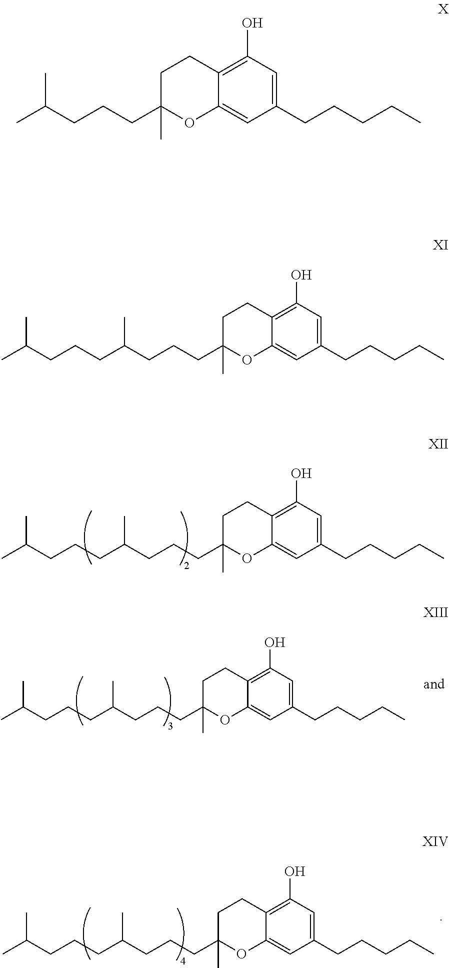

Some aspects of the disclosure are directed to a pharmaceutical composition comprising any of the compounds disclosed herein.

In the context of the present invention, at least the following 43 aspects are described. Aspect 1 is a process for the preparation of a hydrogenated cannabigerol derivative, comprising providing a cannabigerol derivative of formula I to a reaction vessel;

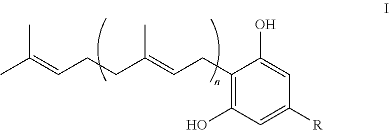

herein n is an integer of from 0 to 4 and R is hydrogen or a substituted or unsubstituted alkyl, alkenyl, or aryl group having from 1 to 10 carbon atoms; providing a catalyst to the reaction vessel; providing hydrogen gas, a source of hydrogen gas, or a combination thereof to the reaction vessel; and producing a hydrogenated cannabigerol derivative of formula II:

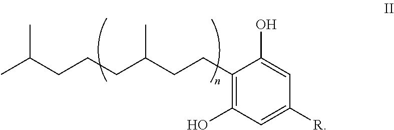

Aspect 2 is the process of Aspect 1, wherein R is methyl, ethyl, propyl, butyl, pentyl, hexyl, heptyl, dimethylheptyl, octyl, phenyl, or benzyl. Aspect 3 is the process of aspect 1, wherein the catalyst is provided in an amount ranging from 0.01 to 10 molar equivalents. Aspect 4 is the process of Aspect 3, wherein the catalyst is selected from the group consisting of Pd/C. Pt/C, Rh/C, Ru/C, Raney nickel, Pd/alumina, Pd/activated charcoal, Pt/alumina, Pt/activated charcoal, Pt₂O (Adam's catalyst), Wilkinson's catalyst ([RhCl(PPh₃)₃]), Crabtree's catalyst ([CsH₁₂IrP(C₆Hₙ)₃CsHsN]PF₆), 9-borabicyclo[3.3.1]nonane, (R/S)-alpine borane, BH₃-DMSO, BH₃-THF, and N-methylimidodiacetic (MIDA) boronates. Aspect 5 is the process of Aspect 4, wherein the catalyst is Pd/C or Pt/C. Aspect 6 is the process of Aspect 1, wherein the hydrogen gas is provided in an amount that affords an intra-vessel gas pressure ranging from 1 bar to 20 bar. Aspect 7 is the process of Aspect 1, wherein hydrogen gas is not provided to the reaction vessel. Aspect 8 is the process of Aspect 1, wherein the source of hydrogen gas generates hydrogen gas in situ. Aspect 9 is the process of Aspect 1, wherein the source of hydrogen gas comprises ammonium formate and/or formic acid. Aspect 10 is the process of Aspect 9, wherein an amount of ammonium formate ranges from 1 to 40 molar equivalents. Aspect 11 is the process of Aspect 9, wherein an amount of formic acid ranges from 1 to 40 molar equivalents. Aspect 12 is the process of Aspect 1, further comprising providing a solvent to the reaction vessel. Aspect 13 is the process of Aspect 12, wherein the solvent is selected from the group consisting of methanol, ethanol, propanol, isopropanol, butanol, tert-butanol, THF. 2-Me-THF, toluene, and ethyl acetate. Aspect 14 is the process of Aspect 1, further comprising heating the reaction vessel. Aspect 15 is the process of Aspect 14, wherein the reaction vessel is heated to a temperature ranging from 25° C. to 100° C. Aspect 16 is the process of Aspect 1, wherein the reaction vessel is hermetically sealed. Aspect 17 is the process of Aspect 1, further comprising purging the reaction vessel with an inert gas prior to addition of reactants and catalyst. Aspect 18 is the process of Aspect 17, wherein the inert gas is nitrogen or argon. Aspect 19 is a process for the preparation of a hydrogenated cannabichromene derivative, comprising providing a cannabichromene derivative of formula III to a reaction vessel

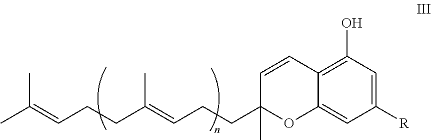

wherein n is an integer of from 0 to 4 and R is hydrogen or a substituted or unsubstituted alkyl, alkenyl, or aryl group having from 1 to 10 carbon atoms; providing a catalyst to the reaction vessel; providing hydrogen gas, a source of hydrogen gas, or a combination thereof to the reaction vessel; and producing a hydrogenated cannabichromene derivative of formula IV

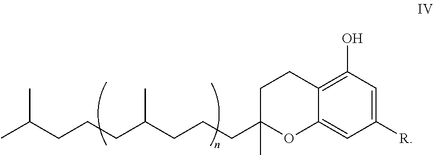

Aspect 20 is the process of Aspect 19, wherein R is methyl, ethyl, propyl, butyl, pentyl, hexyl, heptyl, dimethylheptyl, octyl, phenyl, or benzyl. Aspect 21 is the process of Aspect 19, wherein the catalyst is provided in an amount ranging from 0.01 to 10 molar equivalents. Aspect 22 is the process of Aspect 19, wherein the catalyst is selected from the group consisting of Pd/C, Pt/C, Rh/C, Ru/C, Raney nickel, Pd/alumina, Pd/activated charcoal, Pt/alumina, Pt/activated charcoal, Pt₂O (Adam's catalyst), Wilkinson's catalyst ([RhCl(PPh₃)₃]), Crabtree's catalyst ([CsH₁₂IrP(C₆Hₙ)₃CsHsN]PF₆), 9-borabicyclo[3.3.1]nonane, (R/S)-alpine borane, BH₃-DMSO, BH₃-THF, and N-methylimidodiacetic (MIDA) boronates. Aspect 22 is the process of Aspect 22, wherein the catalyst is Pd/C or Pt/C. Aspect 24 is the process of Aspect 19, wherein the hydrogen gas is provided in an amount that affords an intra-vessel gas pressure ranging from 1 bar to 20 bar. Aspect 25 is the process of Aspect 19, wherein hydrogen gas is not provided to the reaction vessel. Aspect 26 is the process of Aspect 19, wherein the source of hydrogen gas generates hydrogen gas in situ. Aspect 27 is the process of Aspect 19, wherein the source of hydrogen gas comprises ammonium formate and/or formic acid. Aspect 28 is the process of Aspect 27, wherein an amount of ammonium formate ranges from 1 to 40 molar equivalents. Aspect 29 is the process of Aspect 27, wherein an amount of formic acid ranges from 1 to 40 molar equivalents. Aspect 30 is the process of Aspect 19, further comprising providing a solvent to the reaction vessel. Aspect 31 is the process of Aspect 19, wherein the solvent is selected from the group consisting of methanol, ethanol, propanol, isopropanol, butanol, tert-butanol, THF, 2-Me-THF, toluene, and ethyl acetate. Aspect 32 is the process of Aspect 19, further comprising heating the reaction vessel. Aspect 33 is the process of Aspect 33, wherein the reaction vessel is heated to a temperature ranging from 25° C. to 100° C. Aspect 34 is the process of Aspect 19, wherein the reaction vessel is hermetically sealed. Aspect 35 is the process of Aspect 19, further comprising purging the reaction vessel with an inert gas prior to addition of reactants and catalyst. Aspect 36 is the process of Aspect 35, wherein the inert gas is nitrogen or argon. Aspect 37 is a process for the preparation of tetrahydrocannabigerol (H4CBG) comprising providing cannabigerol (CBG) to a reaction vessel; providing a catalyst to the reaction vessel; providing hydrogen gas, a source of hydrogen gas, or a combination thereof to the reaction vessel; and producing H4CBG. Aspect 38 is a process for the preparation of tetrahydrocannabichromene (H4CBC), comprising providing cannabichromene (CBC) to a reaction vessel; providing a catalyst to the reaction vessel; providing hydrogen gas, a source of hydrogen gas, or a combination thereof to the reaction vessel; and producing H4CBC. Aspect 39 is a composition comprising a compound of formula II:

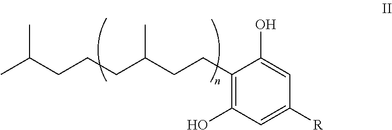

wherein n is an integer of from 0 to 4 and R is hydrogen or a substituted or unsubstituted alkyl, alkenyl, or aryl group having from 1 to 10 carbon atoms. Aspect 40 is the compound of Aspect 39, wherein the compound is further defined as one of

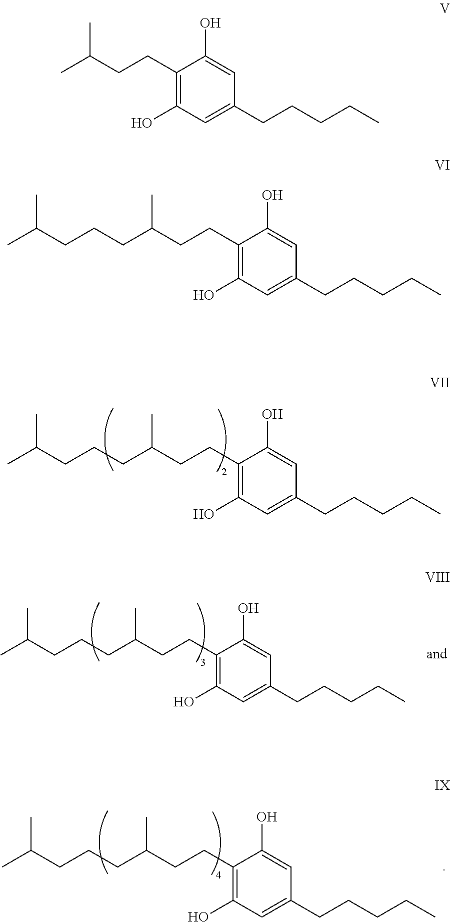

Aspect 41 is a composition comprising a compound of formula IV:

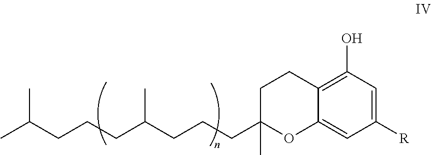

wherein n is an integer of from 0 to 4 and R is hydrogen or a substituted or unsubstituted alkyl, alkenyl, or aryl group having from 1 to 10 carbon atoms. Aspect 42 is the compound of Aspect 42, wherein the compound is further defined as one of:

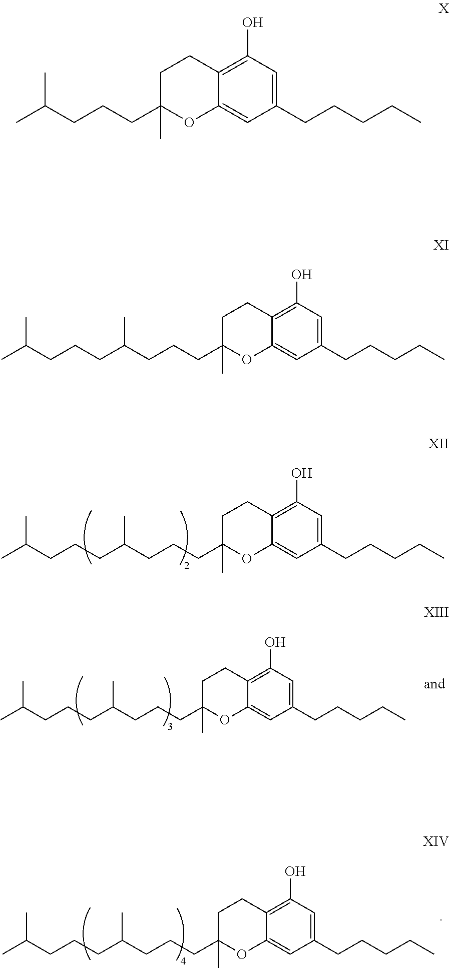

Aspect 43 is a pharmaceutical composition comprising a compound of any of Aspects 37 to 42.

Throughout this application, the term “about” is used to indicate that a value includes the inherent variation of error for the measurement or quantitation method.

The use of the word “a” or “an” when used in conjunction with the term “comprising” may mean “one,” but it is also consistent with the meaning of “one or more,” “at least one,” and “one or more than one.”

The phrase “and/or” means “and” or “or”. To illustrate, A, B, and/or C includes: A alone, B alone, C alone, a combination of A and B, a combination of A and C, a combination of B and C, or a combination of A, B, and C.

The words “comprising” (and any form of comprising, such as “comprise” and “comprises”), “having” (and any form of having, such as “have” and “has”), “including” (and any form of including, such as “includes” and “include”) or “containing” (and any form of containing, such as “contains” and “contain”) are inclusive or open-ended and do not exclude additional, unrecited elements or method steps.

It is contemplated that any embodiment discussed in this specification can be implemented with respect to any method or composition of the invention, and vice versa. Furthermore, compositions of the invention can be used to achieve methods of the invention.

Other objects, features and advantages of the present invention will become apparent from the following detailed description. It should be understood, however, that the detailed description and the specific examples, while indicating specific embodiments of the invention, are given by way of illustration only, since various changes and modifications within the spirit and scope of the invention will become apparent to those skilled in the art from this detailed description.

### BRIEF DESCRIPTION OF THE DRAWINGS

The following drawings form part of the present specification and are included to further demonstrate certain aspects of the present invention. The invention may be better understood by reference to one or more of these drawings in combination with the detailed description of specific embodiments presented herein.

FIGS. 1A-1B Exemplary reaction schemes. FIG. 1A Hydrogenation of a cannagiberol derivative to produce a tetrahydrocannabigerol derivative. FIG. 1B Hydrogenation of a cannabichromene derivative to produce a tetrahydrocannabichromene derivative.

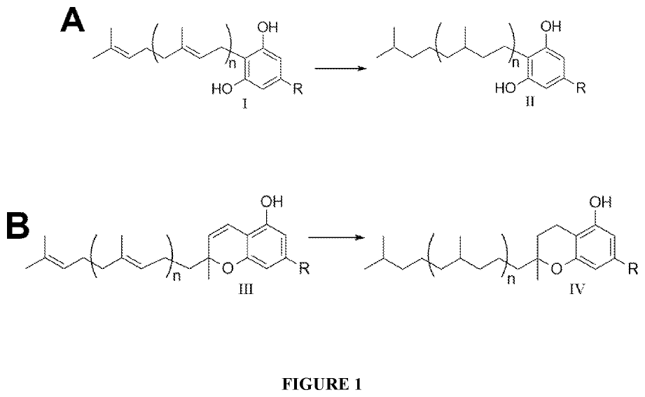

FIGS. 2A-2B Exemplary reaction schemes. FIG. 2A Hydrogenation of cannagiberol to produce tetrahydrocannabigerol. FIG. 2B Hydrogenation of cannabichromene to produce tetrahydrocannabichromene.

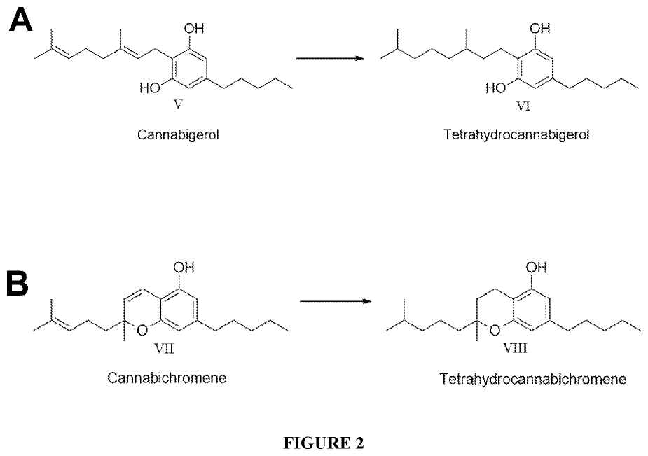

FIGS. 3A-3B HPLC traces. FIG. 3A is an HPLC trace of the tetrahydrocannabigerol product obtained by a method as disclosed herein. FIG. 3B is an HPLC trace of the tetrahydrocannabichromene product obtained by a method as disclosed herein.

FIG. 4 ¹H NMR of the tetrahydrocannabigerol product obtained by a method as disclosed herein.

FIG. 5 ¹³C NMR of the tetrahydrocannabigerol product obtained by a method as disclosed herein.

FIG. 6 ¹H NMR of the tetrahydrocannabichromene product obtained by a method as disclosed herein.

FIG. 7 ¹³C NMR of the tetrahydrocannabichromene product obtained by a method as disclosed herein.

### DETAILED DESCRIPTION OF THE INVENTION

Over 120 different phytocannabinoids have been isolated from Cannabis sativa. Of these, Δ9-tetrahydrocannabinol (Δ9-THC) and cannabidiol (CBD) are the most abundant and widely studied. Given the well-established pharmacological effects of Δ9-THC and CBD, it is not unreasonable to suggest that other phytocannabinoids may exhibit similar or more potent properties.

Cannabichromene (CBC) and cannabigerol (CBG) are phytocannabinoids with relatively low affinity for human CB1 and CB2 receptors, and concomitantly low therapeutic potential. Each of these compounds includes two relatively planar, non-aromatic olefin groups. Conversion of these olefin groups to the corresponding alkyl groups by hydrogenation gives way to multiple related derivatives having more complex tertiary structures. The resulting hydrogenated compounds likely exhibit different affinities for the cannaboid CB1 and CB2 receptors. By applying this method to CBC, CBG, and related derivatives, the present inventors have developed a method for synthesizing a library of hydrogenated CBC and CBG derivatives. These compounds may serve as tools for investigating phytocannabinoid/cannaboid receptor structure activity relationships and may lead to new therapeutics.

### Chemical Definitions

The terms CBG and cannabigerol are used interchangeably herein. The terms H4CBG and tetrahydrocannabigerol are used interchangeably herein. The terms CBC and cannabichromene are used interchangeably herein. The terms H4CBC and tetrahydrocannabichromene are used interchangeably herein. The phrase “semi-synthetic” is defined as a method that employs natural compounds or compounds derived from natural compounds as starting materials to produce different compounds.

As used herein, the term “substituted” is contemplated to include all permissible substituents of organic compounds. In a broad aspect, the permissible substituents include acyclic and cyclic, branched and unbranched, carbocyclic and heterocyclic, and aromatic and nonaromatic substituents of organic compounds. Illustrative substituents include, for example, alkyl, halogenated alkyl, alkoxy, alkenyl, alkynyl, aryl, heteroaryl, aldehyde, amino, carboxylic acid, ester, ether, halide, hydroxy, ketone, nitro, silyl, sulfo-oxo, sulfonyl, sulfone, sulfoxide, and thiol. The permissible substituents can be one or more and the same or different for appropriate organic compounds. For purposes of this disclosure, the heteroatoms, such as nitrogen, can have hydrogen substituents and/or any permissible substituents of organic compounds described herein which satisfy the valencies of the heteroatoms. This disclosure is not intended to be limited in any manner by the permissible substituents of organic compounds. Also, the terms “substitution” or “substituted with” include the implicit proviso that such substitution is in accordance with permitted valence of the substituted atom and the substituent, and that the substitution results in a stable compound, e.g., a compound that does not spontaneously undergo transformation such as by rearrangement, cyclization, elimination, etc.

The term “alkyl” includes straight-chain alkyl, branched-chain alkyl, cycloalkyl (alicyclic), heteroatom-unsubstituted alkyl, heteroatom-substituted alkyl, heteroatom-unsubstituted Cn-alkyl, and heteroatom-substituted Cn-alkyl. In certain embodiments, lower alkyls are contemplated. The term “lower alkyl” refers to alkyls of 1-6 carbon atoms (that is, 1, 2, 3, 4, 5 or 6 carbon atoms). The term “heteroatom-unsubstituted Cn-alkyl” refers to a radical, having a linear or branched, cyclic or acyclic structure, further having no carbon-carbon double or triple bonds, further having a total of n carbon atoms, all of which are nonaromatic, 3 or more hydrogen atoms, and no heteroatoms. For example, a heteroatom-unsubstituted C₁-C₁₀-alkyl has 1 to 10 carbon atoms. The groups, —CH₃ (Me), —CH₂CH₃ (Et), —CH₂CH₂CH₃ (n-Pr), —CH(CH₃)₂ (iso-Pr), —CH(CH₂)₂ (cyclopropyl), —CH₂CH₂CH₂CH₃ (n-Bu), —CH(CH₃)CH₂CH₃ (sec-butyl), —CH₂CH(CH₃)₂ (iso-butyl), —C(CH₃)₃ (tert-butyl), —CH₂C(CH₃)₃ (neo-pentyl), cyclobutyl, cyclopentyl, and cyclohexyl, are all non-limiting examples of heteroatom-unsubstituted alkyl groups. The term “heteroatom-substituted Cn-alkyl” refers to a radical, having a single saturated carbon atom as the point of attachment, no carbon-carbon double or triple bonds, further having a linear or branched, cyclic or acyclic structure, further having a total of n carbon atoms, all of which are nonaromatic, 0, 1, or more than one hydrogen atom, at least one heteroatom, wherein each heteroatom is independently selected from the group consisting of N, O, F, Cl, Br, I, Si, P, and S. For example, a heteroatom-substituted C₁-C₁₀-alkyl has 1 to 10 carbon atoms. The following groups are all non-limiting examples of heteroatom-substituted alkyl groups: trifluoromethyl, —CH₂F, —(CH₂)₂F, —(CH₂)₃F, —(CH₂)₄F, —(CH₂)₅F, —(CH₂)₆F, —(CH₂)₇F, —(CH₂)₈F, —CH₂Cl, —CH₂Br, piperidinyl, —CH₂OH, —CH₂OCH₃, —CH₂OCH₂CF₃, —CH₂OC(O)CH₃, —CH₂NH₂, —CH₂NHCH₃, —CH₂N(CH₃)₂, —CH₂CH₂Cl, —CH₂CH₂OH, CH₂CH₂OC(O)CH₃, —CH₂CH₂NHCO₂C(CH₃)₃, and —CH₂Si(CH₃)₃.

The term “alkenyl” includes straight-chain alkenyl, branched-chain alkenyl, cycloalkenyl, cyclic alkenyl, heteroatom-unsubstituted alkenyl, heteroatom-substituted alkenyl, heteroatom-unsubstituted Cn-alkenyl, and heteroatom-substituted Cn-alkenyl. In certain embodiments, lower alkenyls are contemplated. The term “lower alkenyl” refers to alkenyls of 1-6 carbon atoms (that is, 1, 2, 3, 4, 5 or 6 carbon atoms). The term “heteroatom-unsubstituted Cn-alkenyl” refers to a radical, having a linear or branched, cyclic or acyclic structure, further having at least one nonaromatic carbon-carbon double bond, but no carbon-carbon triple bonds, a total of n carbon atoms, three or more hydrogen atoms, and no heteroatoms. For example, a heteroatom-unsubstituted C₂-C₁₀-alkenyl has 2 to 10 carbon atoms. Heteroatom-unsubstituted alkenyl groups include: —CH═CH₂ (vinyl), —CH═CHCH₃, —CH═CHCH₂CH₃, —CH₂CH═CH₂ (allyl), —CH₂CH═CHCH₃, —CH═CH—C₆H₅, —CH₂CHC(CH₃)₂ (isoprenyl), and —CH₂CHC(CH₃)CH₂(CH₂CHC(CH₃)CH₂)₃CH₂CHC(CH₃)₂ (geranylfarnesyl). The term “heteroatom-substituted Cn-alkenyl” refers to a radical, having a single nonaromatic carbon atom as the point of attachment and at least one nonaromatic carbon-carbon double bond, but no carbon-carbon triple bonds, further having a linear or branched, cyclic or acyclic structure, further having a total of n carbon atoms, 0, 1, or more than one hydrogen atom, and at least one heteroatom, wherein each heteroatom is independently selected from the group consisting of N, O, F, Cl, Br, I, Si, P, and S. For example, a heteroatom-substituted C₂-C₁₀-alkenyl has 2 to 10 carbon atoms. The groups, dihydrofuranyl, —CH═CHF. —CH═CHCl and —CH═CHBr, are non-limiting examples of heteroatom-substituted alkenyl groups.

The term “aryl” includes heteroatom-unsubstituted aryl, heteroatom-substituted aryl, heteroatom-unsubstituted Cn-aryl, heteroatom-substituted Cn-aryl, heteroaryl, heterocyclic aryl groups, carbocyclic aryl groups, biaryl groups, and single-valent radicals derived from polycyclic fused hydrocarbons (PAHs). The term “heteroatom-unsubstituted Cn-aryl” refers to a radical, having a single carbon atom as a point of attachment, wherein the carbon atom is part of an aromatic ring structure containing only carbon atoms, further having a total of n carbon atoms, 5 or more hydrogen atoms, and no heteroatoms. For example, a heteroatom-unsubstituted C₆-C₁₀-aryl has 6 to 10 carbon atoms. Non-limiting examples of heteroatom-unsubstituted aryl groups include phenyl (Ph), methylphenyl, (dimethyl)phenyl, —C₆H₄CH₂CH₃, C₆H₄CH₂CH₂CH₃, C₆H₄CH(CH₃)₂, C₆H₄CH(CH₂)₂, C₆H₃(CH₃)CH₂CH₃, —C₆H₄CH═CH₂, —C₆H₄CH═CHCH₃, —C₆H₄C≡CH, —C₆H₄C≡CCH₃, naphthyl, and the radical derived from biphenyl. The term “heteroatom-substituted Cn-aryl” refers to a radical, having either a single aromatic carbon atom or a single aromatic heteroatom as the point of attachment, further having a total of n carbon atoms, at least one hydrogen atom, and at least one heteroatom, further wherein each heteroatom is independently selected from the group consisting of N, O, F, Cl, Br, I, Si, P, and S. For example, a heteroatom-unsubstituted C₁-C₁₀-heteroaryl has 1 to 10 carbon atoms. Non-limiting examples of heteroatom-substituted aryl groups include the groups: C₆H₄F, —C₆H₄Cl, —C₆H₄Br, —C₆H₄I, —C₆H₄OH, —C₆H₄OCH₃, —C₆H₄OCH₂CH₃, —C₆H₄OC(O)CH₃, —C₆H₄NH₂, —C₆H₄NHCH₃, —C₆H₄N(CH₃)₂, —C₆H₄CH₂OH, —C₆H₄CHOC(O)CH₃, —C₆H₄CH₂NH═, —C₆H₄CF₃, —C₆H₄CN, —C₆H₄CHO, —C₆H₄CHO, —C₆H₄C(O)CH₃, —C₆H₄C(O)C₆H₅, —C₆H₄CO₂H, —C₆H₄CO₂CH₃, —C₆H₄CONH₂, —C₆H₄CONHCH₃, —C₆H₄CON(CH₃)₂, furanyl, thienyl, pyridyl, pyrrolyl, pyrimidyl, pyrazinyl, quinolyl, indolyl, and imidazoyl. In certain embodiments, heteroatom-substituted aryl groups are contemplated. In certain embodiments, heteroatom-unsubstituted aryl groups are contemplate. In certain embodiments, an aryl group may be mono-, di-, tri-, tetra- or penta-substituted with one or more heteroatom-containing substitutents.

The term “aralkyl” includes heteroatom-unsubstituted aralkyl, heteroatom-substituted aralkyl, heteroatom-unsubstituted Cn-aralkyl, heteroatom-substituted Cn-aralkyl, heteroaralkyl, and heterocyclic aralkyl groups. In certain embodiments, lower aralkyls are contemplated. The term “lower aralkyl” refers to aralkyls of 7-12 carbon atoms (that is, 7, 8, 9, 10, 11 or 12 carbon atoms). The term “heteroatom-unsubstituted Cn-aralkyl” refers to a radical, having a single saturated carbon atom as the point of attachment, further having a total of n carbon atoms, wherein at least 6 of the carbon atoms form an aromatic ring structure containing only carbon atoms, 7 or more hydrogen atoms, and no heteroatoms. For example, a heteroatom-unsubstituted C7-C10-aralkyl has 7 to 10 carbon atoms. Non-limiting examples of heteroatom-unsubstituted aralkyls are: phenylmethyl (benzyl, Bn) and phenylethyl. The term “heteroatom-substituted Cn-aralkyl” refers to a radical, having a single saturated carbon atom as the point of attachment, further having a total of n carbon atoms, 0, 1, or more than one hydrogen atom, and at least one heteroatom, wherein at least one of the carbon atoms is incorporated an aromatic ring structures, further wherein each heteroatom is independently selected from the group consisting of N, O, F, Cl, Br, I, Si, P, and S. For example, a heteroatom-substituted C₂-C₁₀-heteroaralkyl has 2 to 10 carbon atoms.

The term “acyl” includes straight-chain acyl, branched-chain acyl, cycloacyl, cyclic acyl, heteroatom-unsubstituted acyl, heteroatom-substituted acyl, heteroatom-unsubstituted Cn-acyl, heteroatom-substituted Cn-acyl, alkylcarbonyl, alkoxycarbonyl and aminocarbonyl groups. In certain embodiments, lower acyls are contemplated. The term “lower acyl” refers to acyls of 1-6 carbon atoms (that is, 1, 2, 3, 4, 5 or 6 carbon atoms). The term “heteroatom-unsubstituted Cn-acyl” refers to a radical, having a single carbon atom of a carbonyl group as the point of attachment, further having a linear or branched, cyclic or acyclic structure, further having a total of n carbon atoms, 1 or more hydrogen atoms, a total of one oxygen atom, and no additional heteroatoms. For example, a heteroatom-unsubstituted C₁-C₁₀-acyl has 1 to 10 carbon atoms. The groups, —CHO, —C(O)CH₃, —C(O)CH₂CH₃, —C(O)CH₂CH₂CH₃, —C(O)CH(CH₃)₂, —C(O)CH(CH₂)₂, —C(O)C₆H₅, —C(O)C₆H₄CH₃, —C(O)C₆H₄CH₂CH₃, and —COC₆H₃(CH₃)₂, are non-limiting examples of heteroatom-unsubstituted acyl groups. The term “heteroatom-substituted Cn-acyl” refers to a radical, having a single carbon atom as the point of attachment, the carbon atom being part of a carbonyl group, further having a linear or branched, cyclic or acyclic structure, further having a total of n carbon atoms, 0, 1, or more than one hydrogen atom, at least one additional heteroatom, in addition to the oxygen of the carbonyl group, wherein each additional heteroatom is independently selected from the group consisting of N, O, F, Cl, Br, I, Si, P, and S. For example, a heteroatom-substituted C₁-C₁₀-acyl has 1 to 10 carbon atoms. The groups, —C(O)CH₂CF₃, —CO₂H, —CO₂—, —CO₂CH₃, —CO₂CH₂CH₃, —CO₂CH₂CH₂CH₃, —CO₂CH(CH₃)₂, —CO₂CH(CH₂)₂, —C(O) NH₂ (carbamoyl), —C(O)NHCH₃, —C(O)NHCH₂CH₃, —CONHCH(CH₃)₂, —CONHCH(CH₂)₂, —CON(CH₃)₂, and —CONHCH₂CF₃, are non-limiting examples of heteroatom-substituted acyl groups.

The term “alkoxy” includes straight-chain alkoxy, branched-chain alkoxy, cycloalkoxy, cyclic alkoxy, heteroatom-unsubstituted alkoxy, heteroatom-substituted alkoxy, heteroatom-unsubstituted Cn-alkoxy, and heteroatom-substituted Cn-alkoxy. In certain embodiments, lower alkoxys are contemplated. The term “lower alkoxy” refers to alkoxys of 1-6 carbon atoms (that is, 1, 2, 3, 4, 5 or 6 carbon atoms). The term “heteroatom-unsubstituted Cn-alkoxy” refers to a group, having the structure —OR, in which R is a heteroatom-unsubstituted Cn-alkyl, as that term is defined above. Heteroatom-unsubstituted alkoxy groups include: —OCH₃, —OCH₂CH₃, —OCH₂CH₂CH₃, —OCH(CH₃)₂, and —OCH(CH₂)₂. The term “heteroatom-substituted Cn-alkoxy” refers to a group, having the structure —OR, in which R is a heteroatom-substituted Cn-alkyl, as that term is defined above. For example, —OCH₂CF₃ is a heteroatom-substituted alkoxy group.

The term “alkenyloxy” includes straight-chain alkenyloxy, branched-chain alkenyloxy, cycloalkenyloxy, cyclic alkenyloxy, heteroatom-unsubstituted alkenyloxy, heteroatom-substituted alkenyloxy, heteroatom-unsubstituted Cn-alkenyloxy, and heteroatom-substituted Cn-alkenyloxy. The term “heteroatom-unsubstituted Cn-alkenyloxy” refers to a group, having the structure —OR, in which R is a heteroatom-unsubstituted Cn-alkenyl, as that term is defined above. The term “heteroatom-substituted Cn-alkenyloxy” refers to a group, having the structure —OR, in which R is a heteroatom-substituted Cn-alkenyl, as that term is defined above.

The term “alkynyloxy” includes straight-chain alkynyloxy, branched-chain alkynyloxy, cycloalkynyloxy, cyclic alkynyloxy, heteroatom-unsubstituted alkynyloxy, heteroatom-substituted alkynyloxy, heteroatom-unsubstituted Cn-alkynyloxy, and heteroatom-substituted Cn-alkynyloxy. The term “heteroatom-unsubstituted Cn-alkynyloxy” refers to a group, having the structure —OR, in which R is a heteroatom-unsubstituted Cn-alkynyl, as that term is defined above. The term “heteroatom-substituted Cn-alkynyloxy” refers to a group, having the structure —OR, in which R is a heteroatom-substituted Cn-alkynyl, as that term is defined above.

The term “aryloxy” includes heteroatom-unsubstituted aryloxy, heteroatom-substituted aryloxy, heteroatom-unsubstituted Cn-aryloxy, heteroatom-substituted Cn-aryloxy, heteroaryloxy, and heterocyclic aryloxy groups. The term “heteroatom-unsubstituted Cn-aryloxy” refers to a group, having the structure —OAr, in which Ar is a heteroatom-unsubstituted Cn-aryl, as that term is defined above. A non-limiting example of a heteroatom-unsubstituted aryloxy group is —OC₆H₅. The term “heteroatom-substituted Cn-aryloxy” refers to a group, having the structure —OAr, in which Ar is a heteroatom-substituted Cn-aryl, as that term is defined above.

The term “aralkyloxy” includes heteroatom-unsubstituted aralkyloxy, heteroatom-substituted aralkyloxy, heteroatom-unsubstituted Cn-aralkyloxy, heteroatom-substituted Cn-aralkyloxy, heteroaralkyloxy, and heterocyclic aralkyloxy groups. The term “heteroatom-unsubstituted Cn-aralkyloxy” refers to a group, having the structure —OAr, in which Ar is a heteroatom-unsubstituted Cn-aralkyl, as that term is defined above. The term “heteroatom-substituted Cn-aralkyloxy” refers to a group, having the structure —OAr, in which Ar is a heteroatom-substituted Cn-aralkyl, as that term is defined above.

The term “acyloxy” includes straight-chain acyloxy, branched-chain acyloxy, cycloacyloxy, cyclic acyloxy, heteroatom-unsubstituted acyloxy, heteroatom-substituted acyloxy, heteroatom-unsubstituted Cn-acyloxy, heteroatom-substituted Cn-acyloxy, alkylcarbonyloxy, arylcarbonyloxy, alkoxycarbonyloxy, aryloxycarbonyloxy, and carboxylate groups. The term “heteroatom-unsubstituted Cn-acyloxy” refers to a group, having the structure —OAc, in which Ac is a heteroatom-unsubstituted Cn-acyl, as that term is defined above. For example, —OC(O)CH₃ is a non-limiting example of a heteroatom-unsubstituted acyloxy group. The term “heteroatom-substituted Cn-acyloxy” refers to a group, having the structure —OAc, in which Ac is a heteroatom-substituted Cn-acyl, as that term is defined above. For example, —OC(O) OCH₃ and —OC(O)NHCH₃ are non-limiting examples of heteroatom-unsubstituted acyloxy groups.

The term “alkylamino” includes straight-chain alkylamino, branched-chain alkylamino, cycloalkylamino, cyclic alkylamino, heteroatom-unsubstituted alkylamino, heteroatom-substituted alkylamino, heteroatom-unsubstituted Cn-alkylamino, and heteroatom-substituted Cn-alkylamino. The term “heteroatom-unsubstituted Cn-alkylamino” refers to a radical, having a single nitrogen atom as the point of attachment, further having one or two saturated carbon atoms attached to the nitrogen atom, further having a linear or branched, cyclic or acyclic structure, containing a total of n carbon atoms, all of which are nonaromatic, 4 or more hydrogen atoms, a total of 1 nitrogen atom, and no additional heteroatoms. For example, a heteroatom-unsubstituted C₁-C₁₀-alkylamino has 1 to 10 carbon atoms. The term “heteroatom-unsubstituted Cn-alkylamino” includes groups, having the structure —NHR, in which R is a heteroatom-unsubstituted Cn-alkyl, as that term is defined above. A heteroatom-unsubstituted alkylamino group would include —NHCH₃, —NHCH₂CH₃, —NHCH₂CH₂CH₃, —NHCH(CH₃)₂, —NHCH(CH₂)₂, —NHCH₂CH₂CH₂CH₃, —NHCH(CH₃)CH₂CH₃, —NHCH₂CH(CH₃)₂, —NHC(CH₃)₃, —N(CH₃)₂, —N(CH₃)CH₂CH₃, —N(CH₂CH₃)₂, N-pyrrolidinyl, and N-piperidinyl. The term “heteroatom-substituted Cn-alkylamino” refers to a radical, having a single nitrogen atom as the point of attachment, further having one or two saturated carbon atoms attached to the nitrogen atom, no carbon-carbon double or triple bonds, further having a linear or branched, cyclic or acyclic structure, further having a total of n carbon atoms, all of which are nonaromatic, 0, 1, or more than one hydrogen atom, and at least one additional heteroatom, that is, in addition to the nitrogen atom at the point of attachment, wherein each additional heteroatom is independently selected from the group consisting of N, O, F, Cl, Br, I, Si, P, and S. For example, a heteroatom-substituted C₁-C₁₀-alkylamino has 1 to 10 carbon atoms. The term “heteroatom-substituted Cn-alkylamino” includes groups, having the structure —NHR, in which R is a heteroatom-substituted Cn-alkyl, as that term is defined above.

The term “alkenylamino” includes straight-chain alkenylamino, branched-chain alkenylamino, cycloalkenylamino, cyclic alkenylamino, heteroatom-unsubstituted alkenylamino, heteroatom-substituted alkenylamino, heteroatom-unsubstituted Cn-alkenylamino, heteroatom-substituted Cn-alkenylamino, dialkenylamino, and alkyl(alkenyl)amino groups. The term “heteroatom-unsubstituted Cn-alkenylamino” refers to a radical, having a single nitrogen atom as the point of attachment, further having one or two carbon atoms attached to the nitrogen atom, further having a linear or branched, cyclic or acyclic structure, containing at least one nonaromatic carbon-carbon double bond, a total of n carbon atoms, 4 or more hydrogen atoms, a total of one nitrogen atom, and no additional heteroatoms. For example, a heteroatom-unsubstituted C₂-C₁₀-alkenylamino has 2 to 10 carbon atoms. The term “heteroatom-unsubstituted Cn-alkenylamino” includes groups, having the structure —NHR, in which R is a heteroatom-unsubstituted Cn-alkenyl, as that term is defined above. The term “heteroatom-substituted Cn-alkenylamino” refers to a radical, having a single nitrogen atom as the point of attachment and at least one nonaromatic carbon-carbon double bond, but no carbon-carbon triple bonds, further having one or two carbon atoms attached to the nitrogen atom, further having a linear or branched, cyclic or acyclic structure, further having a total of n carbon atoms, 0, 1, or more than one hydrogen atom, and at least one additional heteroatom, that is, in addition to the nitrogen atom at the point of attachment, wherein each additional heteroatom is independently selected from the group consisting of N, O, F, Cl, Br, I, Si, P, and S. For example, a heteroatom-substituted C₂-C₁₀-alkenylamino has 2 to 10 carbon atoms. The term “heteroatom-substituted Cn-alkenylamino” includes groups, having the structure —NHR, in which R is a heteroatom-substituted Cn-alkenyl, as that term is defined above.

The term “alkynylamino” includes straight-chain alkynylamino, branched-chain alkynylamino, cycloalkynylamino, cyclic alkynylamino, heteroatom-unsubstituted alkynylamino, heteroatom-substituted alkynylamino, heteroatom-unsubstituted Cn-alkynylamino, heteroatom-substituted Cn-alkynylamino, dialkynylamino, alkyl(alkynyl)amino, and alkenyl(alkynyl)amino groups. The term “heteroatom-unsubstituted Cn-alkynylamino” refers to a radical, having a single nitrogen atom as the point of attachment, further having one or two carbon atoms attached to the nitrogen atom, further having a linear or branched, cyclic or acyclic structure, containing at least one carbon-carbon triple bond, a total of n carbon atoms, at least one hydrogen atoms, a total of one nitrogen atom, and no additional heteroatoms. For example, a heteroatom-unsubstituted C₂-C₁₀-alkynylamino has 2 to 10 carbon atoms. The term “heteroatom-unsubstituted Cn-alkynylamino” includes groups, having the structure —NHR, in which R is a heteroatom-unsubstituted Cn-alkynyl, as that term is defined above. The term “heteroatom-substituted Cn-alkynylamino” refers to a radical, having a single nitrogen atom as the point of attachment, further having one or two carbon atoms attached to the nitrogen atom, further having at least one nonaromatic carbon-carbon triple bond, further having a linear or branched, cyclic or acyclic structure, and further having a total of n carbon atoms, 0, 1, or more than one hydrogen atom, and at least one additional heteroatom, that is, in addition to the nitrogen atom at the point of attachment, wherein each additional heteroatom is independently selected from the group consisting of N, O, F, Cl, Br, I, Si, P, and S. For example, a heteroatom-substituted C₂-C₁₀-alkynylamino has 2 to 10 carbon atoms. The term “heteroatom-substituted Cn-alkynylamino” includes groups, having the structure —NHR, in which R is a heteroatom-substituted Cn-alkynyl, as that term is defined above.

The term “arylamino” includes heteroatom-unsubstituted arylamino, heteroatom-substituted arylamino, heteroatom-unsubstituted Cn-arylamino, heteroatom-substituted Cn-arylamino, heteroarylamino, heterocyclic arylamino, and alkyl(aryl)amino groups. The term “heteroatom-unsubstituted Cn-arylamino” refers to a radical, having a single nitrogen atom as the point of attachment, further having at least one aromatic ring structure attached to the nitrogen atom, wherein the aromatic ring structure contains only carbon atoms, further having a total of n carbon atoms, 6 or more hydrogen atoms, a total of one nitrogen atom, and no additional heteroatoms. For example, a heteroatom-unsubstituted C₆-C₁₀-arylamino has 6 to 10 carbon atoms. The term “heteroatom-unsubstituted Cn-arylamino” includes groups, having the structure —NHR, in which R is a heteroatom-unsubstituted Cn-aryl, as that term is defined above. The term “heteroatom-substituted Cn-arylamino” refers to a radical, having a single nitrogen atom as the point of attachment, further having a total of n carbon atoms, at least one hydrogen atom, at least one additional heteroatoms, that is, in addition to the nitrogen atom at the point of attachment, wherein at least one of the carbon atoms is incorporated into one or more aromatic ring structures, further wherein each additional heteroatom is independently selected from the group consisting of N. O. F, Cl, Br, I, Si, P, and S. For example, a heteroatom-substituted C₆-C₁₀-arylamino has 6 to 10 carbon atoms. The term “heteroatom-substituted Cn-arylamino” includes groups, having the structure —NHR, in which R is a heteroatom-substituted Cn-aryl, as that term is defined above.

The term “aralkylamino” includes heteroatom-unsubstituted aralkylamino, heteroatom-substituted aralkylamino, heteroatom-unsubstituted Cn-aralkylamino, heteroatom-substituted Cn-aralkylamino, heteroaralkylamino, heterocyclic aralkylamino groups, and diaralkylamino groups. The term “heteroatom-unsubstituted Cn-aralkylamino” refers to a radical, having a single nitrogen atom as the point of attachment, further having one or two saturated carbon atoms attached to the nitrogen atom, further having a total of n carbon atoms, wherein at least 6 of the carbon atoms form an aromatic ring structure containing only carbon atoms, 8 or more hydrogen atoms, a total of one nitrogen atom, and no additional heteroatoms. For example, a heteroatom-unsubstituted C₇-C₁₀-aralkylamino has 7 to 10 carbon atoms. The term “heteroatom-unsubstituted Cn-aralkylamino” includes groups, having the structure-NHR, in which R is a heteroatom-unsubstituted Cn-aralkyl, as that term is defined above. The term “heteroatom-substituted Cn-aralkylamino” refers to a radical, having a single nitrogen atom as the point of attachment, further having at least one or two saturated carbon atoms attached to the nitrogen atom, further having a total of n carbon atoms, 0, 1, or more than one hydrogen atom, at least one additional heteroatom, that is, in addition to the nitrogen atom at the point of attachment, wherein at least one of the carbon atom incorporated into an aromatic ring, further wherein each heteroatom is independently selected from the group consisting of N, O, F, Cl, Br, I, Si, P, and S. For example, a heteroatom-substituted C₇-C₁₀-aralkylamino has 7 to 10 carbon atoms. The term “heteroatom-substituted Cn-aralkylamino” includes groups, having the structure —NHR, in which R is a heteroatom-substituted Cn-aralkyl, as that term is defined above.

The term “amido” includes straight-chain amido, branched-chain amido, cycloamido, cyclic amido, heteroatom-unsubstituted amido, heteroatom-substituted amido, heteroatom-unsubstituted Cn-amido, heteroatom-substituted Cn-amido, alkylcarbonylamino, arylcarbonylamino, alkoxycarbonylamino, aryloxycarbonylamino, acylamino, alkylaminocarbonylamino, arylaminocarbonylamino, and ureido groups. The term “heteroatom-unsubstituted Cn-amido” refers to a radical, having a single nitrogen atom as the point of attachment, further having a carbonyl group attached via its carbon atom to the nitrogen atom, further having a linear or branched, cyclic or acyclic structure, further having a total of n carbon atoms, 1 or more hydrogen atoms, a total of one oxygen atom, a total of one nitrogen atom, and no additional heteroatoms. For example, a heteroatom-unsubstituted C₁-C₁₀-amido has 1 to 10 carbon atoms. The term “heteroatom-unsubstituted Cn-amido” includes groups, having the structure —NHR, in which R is a heteroatom-unsubstituted Cn-acyl, as that term is defined above. The group, —NHC(O)CH₃, is a non-limiting example of a heteroatom-unsubstituted amido group. The term “heteroatom-substituted Cn-amido” refers to a radical, having a single nitrogen atom as the point of attachment, further having a carbonyl group attached via its carbon atom to the nitrogen atom, further having a linear or branched, cyclic or acyclic structure, further having a total of n aromatic or nonaromatic carbon atoms, 0, 1, or more than one hydrogen atom, at least one additional heteroatom in addition to the oxygen of the carbonyl group, wherein each additional heteroatom is independently selected from the group consisting of N, O, F, Cl, Br, I, Si, P, and S. For example, a heteroatom-substituted C₁-C₁₀-amido has 1 to 10 carbon atoms. The term “heteroatom-substituted Cn-amido” includes groups, having the structure —NHR, in which R is a heteroatom-unsubstituted Cn-acyl, as that term is defined above. The group, —NHCO₂CH₃, is a non-limiting example of a heteroatom-substituted amido group.

The term “alkylthio” includes straight-chain alkylthio, branched-chain alkylthio, cycloalkylthio, cyclic alkylthio, heteroatom-unsubstituted alkylthio, heteroatom-substituted alkylthio, heteroatom-unsubstituted Cn-alkylthio, and heteroatom-substituted Cn-alkylthio. The term “heteroatom-unsubstituted Cn-alkylthio” refers to a group, having the structure-SR, in which R is a heteroatom-unsubstituted Cn-alkyl, as that term is defined above. The group, —SCH₃, is an example of a heteroatom-unsubstituted alkylthio group. The term “heteroatom-substituted Cn-alkylthio” refers to a group, having the structure —SR, in which R is a heteroatom-substituted Cn-alkyl, as that term is defined above.

The term “alkenylthio” includes straight-chain alkenylthio, branched-chain alkenylthio, cycloalkenylthio, cyclic alkenylthio, heteroatom-unsubstituted alkenylthio, heteroatom-substituted alkenylthio, heteroatom-unsubstituted Cn-alkenylthio, and heteroatom-substituted Cn-alkenylthio. The term “heteroatom-unsubstituted Cn-alkenylthio” refers to a group, having the structure —SR, in which R is a heteroatom-unsubstituted Cn-alkenyl, as that term is defined above. The term “heteroatom-substituted Cn-alkenylthio” refers to a group, having the structure —SR, in which R is a heteroatom-substituted Cn-alkenyl, as that term is defined above.

The term “alkynylthio” includes straight-chain alkynylthio, branched-chain alkynylthio, cycloalkynylthio, cyclic alkynylthio, heteroatom-unsubstituted alkynylthio, heteroatom-substituted alkynylthio, heteroatom-unsubstituted Cn-alkynylthio, and heteroatom-substituted Cn-alkynylthio. The term “heteroatom-unsubstituted Cn-alkynylthio” refers to a group, having the structure —SR, in which R is a heteroatom-unsubstituted Cn-alkynyl, as that term is defined above. The term “heteroatom-substituted Cn-alkynylthio” refers to a group, having the structure —SR, in which R is a heteroatom-substituted Cn-alkynyl, as that term is defined above.

The term “arylthio” includes heteroatom-unsubstituted arylthio, heteroatom-substituted arylthio, heteroatom-unsubstituted Cn-arylthio, heteroatom-substituted Cn-arylthio, heteroarylthio, and heterocyclic arylthio groups. The term “heteroatom-unsubstituted Cn-arylthio” refers to a group, having the structure —SAr, in which Ar is a heteroatom-unsubstituted Cn-aryl, as that term is defined above. The group, —SC₆H₅, is an example of a heteroatom-unsubstituted arylthio group. The term “heteroatom-substituted Cn-arylthio” refers to a group, having the structure —SAr, in which Ar is a heteroatom-substituted Cn-aryl, as that term is defined above.

The term “aralkylthio” includes heteroatom-unsubstituted aralkylthio, heteroatom-substituted aralkylthio, heteroatom-unsubstituted Cn-aralkylthio, heteroatom-substituted Cn-aralkylthio, heteroaralkylthio, and heterocyclic aralkylthio groups. The term “heteroatom-unsubstituted Cn-aralkylthio” refers to a group, having the structure —SAr, in which Ar is a heteroatom-unsubstituted Cn-aralkyl, as that term is defined above. The group, —SCH₂C₆H₅, is an example of a heteroatom-unsubstituted aralkyl group. The term “heteroatom-substituted Cn-aralkylthio” refers to a group, having the structure —SAr, in which Ar is a heteroatom-substituted Cn-aralkyl, as that term is defined above.

The term “acylthio” includes straight-chain acylthio, branched-chain acylthio, cycloacylthio, cyclic acylthio, heteroatom-unsubstituted acylthio, heteroatom-substituted acylthio, heteroatom-unsubstituted Cn-acylthio, heteroatom-substituted Cn-acylthio, alkylcarbonyloxy, arylcarbonyloxy, alkoxycarbonyloxy, aryloxycarbonyloxy, and carboxylate groups. The term “heteroatom-unsubstituted Cn-acylthio” refers to a group, having the structure —SAc, in which Ac is a heteroatom-unsubstituted Cn-acyl, as that term is defined above. The group, —SCOCH₃, is an example of a heteroatom-unsubstituted acylthio group. The term “heteroatom-substituted Cn-acylthio” refers to a group, having the structure —SAc, in which Ac is a heteroatom-substituted Cn-acyl, as that term is defined above.

The term “alkylsilyl” includes straight-chain alkylsilyl, branched-chain alkylsilyl, cycloalkylsilyl, cyclic alkylsilyl, heteroatom-unsubstituted alkylsilyl, heteroatom-substituted alkylsilyl, heteroatom-unsubstituted Cn-alkylsilyl, and heteroatom-substituted Cn-alkylsilyl. The term “heteroatom-unsubstituted Cn-alkylsilyl” refers to a radical, having a single silicon atom as the point of attachment, further having one, two, or three saturated carbon atoms attached to the silicon atom, further having a linear or branched, cyclic or acyclic structure, containing a total of n carbon atoms, all of which are nonaromatic, 5 or more hydrogen atoms, a total of 1 silicon atom, and no additional heteroatoms. For example, a heteroatom-unsubstituted C₁-C₁₀-alkylsilyl has 1 to 10 carbon atoms. An alkylsilyl group includes dialkylamino groups. The groups, —Si(CH₃)₃ and —Si(CH₃)₂C(CH₃)₃, are non-limiting examples of heteroatom-unsubstituted alkylsilyl groups. The term “heteroatom-substituted Cn-alkylsilyl” refers to a radical, having a single silicon atom as the point of attachment, further having at least one, two, or three saturated carbon atoms attached to the silicon atom, no carbon-carbon double or triple bonds, further having a linear or branched, cyclic or acyclic structure, further having a total of n carbon atoms, all of which are nonaromatic, 0, 1, or more than one hydrogen atom, and at least one additional heteroatom, that is, in addition to the silicon atom at the point of attachment, wherein each additional heteroatom is independently selected from the group consisting of N, O, F, Cl, Br, I, Si, P, and S. For example, a heteroatom-substituted C₁-C₁₀-alkylsilyl has 1 to 10 carbon atoms.

The term “phosphonate” includes straight-chain phosphonate, branched-chain phosphonate, cyclophosphonate, cyclic phosphonate, heteroatom-unsubstituted phosphonate, heteroatom-substituted phosphonate, heteroatom-unsubstituted Cn-phosphonate, and heteroatom-substituted Cn-phosphonate. The term “heteroatom-unsubstituted Cn-phosphonate” refers to a radical, having a single phosphorous atom as the point of attachment, further having a linear or branched, cyclic or acyclic structure, further having a total of n carbon atoms, 2 or more hydrogen atoms, a total of three oxygen atom, and no additional heteroatoms. The three oxygen atoms are directly attached to the phosphorous atom, with one of these oxygen atoms doubly bonded to the phosphorous atom. For example, a heteroatom-unsubstituted C₀-C₁₀-phosphonate has 0 to 10 carbon atoms. The groups, —P(O)(OH)₂, —P(O)(OH)OCH₃, —P(O)(OH)OCH₂CH₃, —P(O)(OCH₃)₂, and —P(O)(OH)(OCH₅) are non-limiting examples of heteroatom-unsubstituted phosphonate groups. The term “heteroatom-substituted Cn-phosphonate” refers to a radical, having a single phosphorous atom as the point of attachment, further having a linear or branched, cyclic or acyclic structure, further having a total of n carbon atoms, 2 or more hydrogen atoms, three or more oxygen atoms, three of which are directly attached to the phosphorous atom, with one of these three oxygen atoms doubly bonded to the phosphorous atom, and further having at least one additional heteroatom in addition to the three oxygen atoms, wherein each additional heteroatom is independently selected from the group consisting of N, O, F, Cl, Br, I, Si, P, and S. For example, a heteroatom-unsubstituted C₀-C₁₀-phosphonate has 0 to 10 carbon atoms.

The term “phosphinate” includes straight-chain phosphinate, branched-chain phosphinate, cyclophosphinate, cyclic phosphinate, heteroatom-unsubstituted phosphinate, heteroatom-substituted phosphinate, heteroatom-unsubstituted Cn-phosphinate, and heteroatom-substituted Cn-phosphinate. The term “heteroatom-unsubstituted Cn-phosphinate” refers to a radical, having a single phosphorous atom as the point of attachment, further having a linear or branched, cyclic or acyclic structure, further having a total of n carbon atoms, 2 or more hydrogen atoms, a total of two oxygen atom, and no additional heteroatoms. The two oxygen atoms are directly attached to the phosphorous atom, with one of these oxygen atoms doubly bonded to the phosphorous atom. For example, a heteroatom-unsubstituted C₀-C₁₀-phosphinate has 0 to 10 carbon atoms. The groups, —P(O)(OH) H, —P(O)(OH)CH₃, —P(O)(OH)CH₂CH₃, —P(O)(OCH₃)CH₃, and —P(O)(OC₆H₅) H are non-limiting examples of heteroatom-unsubstituted phosphinate groups. The term “heteroatom-substituted Cn-phosphinate” refers to a radical, having a single phosphorous atom as the point of attachment, further having a linear or branched, cyclic or acyclic structure, further having a total of n carbon atoms, 2 or more hydrogen atoms, two or more oxygen atoms, two of which are directly attached to the phosphorous atom, with one of these two oxygen atoms doubly bonded to the phosphorous atom, and further having at least one additional heteroatom in addition to the two oxygen atoms, wherein each additional heteroatom is independently selected from the group consisting of N, O, F, Cl, Br, I, Si, P, and S. For example, a heteroatom-unsubstituted C₀-C₁₀-phosphinate has 0 to 10 carbon atoms.

Unless stated to the contrary, a formula with chemical bonds shown only as solid lines and not as wedges or dashed lines contemplates each possible isomer, e.g., each enantiomer, diastereomer, and meso compound, and a mixture of isomers, such as a racemic or scalemic mixture.

The claimed invention is also intended to encompass salts of any of the compounds of the present invention. The term “salt(s)” as used herein, is understood as being acidic and/or basic salts formed with inorganic and/or organic acids and bases. Zwitterions (internal or inner salts) are understood as being included within the term “salt(s)” as used herein, as are quaternary ammonium salts such as alkylammonium salts. Nontoxic, pharmaceutically acceptable salts are preferred, although other salts may be useful, as for example in isolation or purification steps during synthesis. Salts include, but are not limited to, sodium, lithium, potassium, amines, tartrates, citrates, hydrohalides, phosphates and the like. A salt may be a pharmaceutically acceptable salt, for example. Thus, pharmaceutically acceptable salts of compounds of the present invention are contemplated.

The term “pharmaceutically acceptable salts,” as used herein, refers to salts of compounds of this invention that are substantially non-toxic to living organisms. Typical pharmaceutically acceptable salts include those salts prepared by reaction of a compound of this invention with an inorganic or organic acid, or an organic base, depending on the substituents present on the compounds of the invention.

Compounds employed in methods of the invention may contain one or more asymmetrically-substituted carbon or nitrogen atoms, and may be isolated in optically active or racemic form. Thus, all chiral, diastereomeric, racemic form, epimeric form, and all geometric isomeric forms of a structure are intended, unless the specific stercochemistry or isomeric form is specifically indicated. Compounds may occur as racemates and racemic mixtures, single enantiomers, diastereomeric mixtures and individual diastereomers. In some embodiments, a single diastereomer is obtained. The chiral centers of the compounds of the present invention can have the S- or the R-configuration, as defined by the IUPAC 1974 Recommendations. Compounds may be of the D- or L-form, for example. It is well known in the art how to prepare and isolate such optically active forms. For example, mixtures of stereoisomers may be separated by standard techniques including, but not limited to, resolution of racemic form, normal, reverse-phase, and chiral chromatography, preferential salt formation, recrystallization, and the like, or by chiral synthesis either from chiral starting materials or by deliberate synthesis of target chiral centers.

In addition, atoms making up the compounds of the present invention are intended to include all isotopic forms of such atoms. Isotopes, as used herein, include those atoms having the same atomic number but different mass numbers. By way of general example and without limitation, isotopes of hydrogen include tritium and deuterium, and isotopes of carbon include ¹³C and ¹⁴C.

Various forms of palladium catalyst useful for the reaction are discussed by Blaser et. al., Supported palladium catalysts for fine chemicals synthesis in Journal of Molecular Catalysis A: Chemical, 2001, v. 172, p. 3-18, the entirety of which is incorporated by reference.

### EXAMPLES

The following examples are included to demonstrate preferred embodiments of the invention. It should be appreciated by those of skill in the art that the techniques disclosed in the examples which follow represent techniques discovered by the inventor to function well in the practice of the invention, and thus can be considered to constitute preferred modes for its practice. However, those of skill in the art should, in light of the present disclosure, appreciate that many changes can be made in the specific embodiments which are disclosed and still obtain a like or similar result without departing from the spirit and scope of the invention.

### Example 1

### Synthesis of Hydrogenated CBG Using Hydrogen Gas

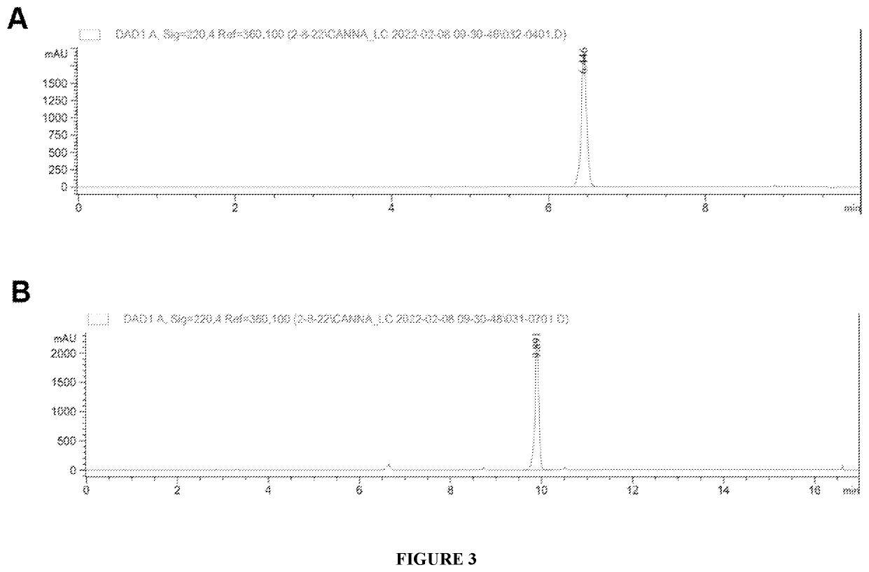

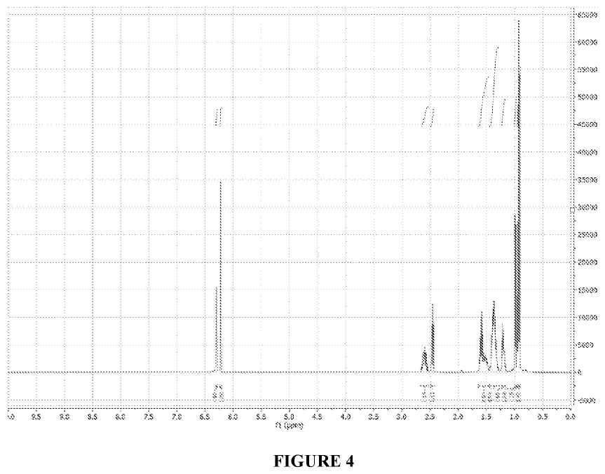

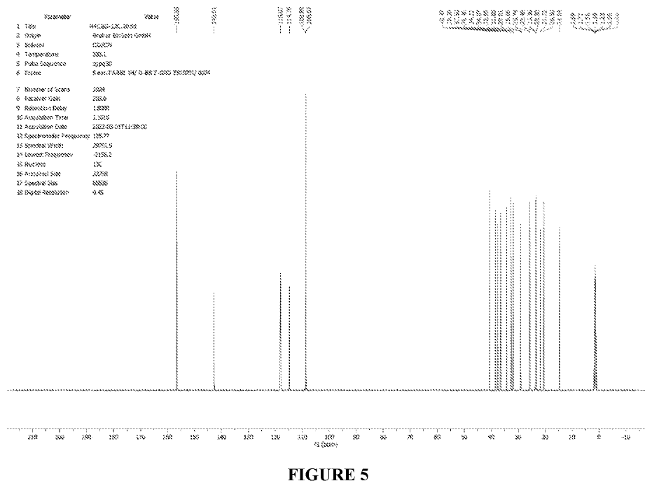

CBG was added to a polar protic or polar aprotic solvent in a reaction flask. A metal catalyst was added to the reaction flask under argon. A hydrogen source was added under argon slowly so as to avoid bumping of the solution. The reaction mixture was stirred until the reaction was observed to have been completed by HPLC. H₄CBG is a white solid, M.P.: 73.8° C. FIG. 3A HPLC C₁₈: 6.446 min, FIG. 4 ¹H NMR (500 MHZ, MeCN): 6.30 (1H), 6.23 (1H), 2.60 (1H), 2.46 (1H), 1.55 (3H), 1.36 (5H), 1.20 (2H), 0.98 (2H), 0.95 (1H), 0.92 (3H). FIG. 5 ¹³C NMR.

### Example 2

### Synthesis of Hydrogenated CBG Using an Additive

To a two-neck round bottomed flask equipped with a reflux condenser was added CBG (2 grams) and the flask was charged with methanol (50-300 mL). The flask was purged of air using vacuum. The flask was then filled with argon. The purge/fill cycle was then repeated three times total. Afterwards, the additive ammonium formate (1 to 20 molar equivalents) was slowly added to the round bottomed flask. Pd/C (0.1 to 5 molar equivalent by percentage of Palladium loading) was then added slowly to the reaction mixture by way of a powder funnel. The reaction was then stirred until the reaction was observed to have been completed using HPLC as a guide. Once complete, the reaction mixture was then filtered over celite to remove the Pd/C. The mixture was then placed onto a roto evaporator to remove all methanol. It was then dissolved in hexane. The reaction mixture dissolved in hexane was then washed with water (10-100 mL, 3 times) in a separatory funnel. The aqueous layer was removed after each wash. The organic layer was then washed with a saturated brine solution (10-100 mL) and the aqueous layer was removed. The organic layer was then concentrated in vacuo. This yellow oil was then be purified via crystallization in alkane solvents to afford a white crystalline powder.

### Example 3

### Synthesis of Hydrogenated CBC

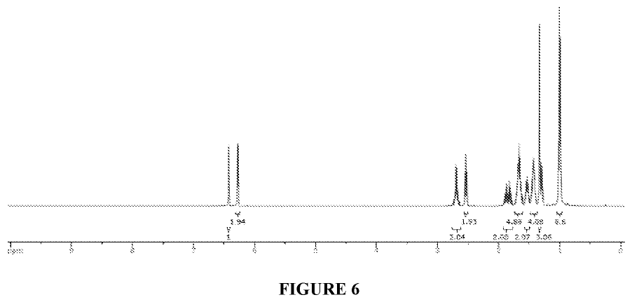

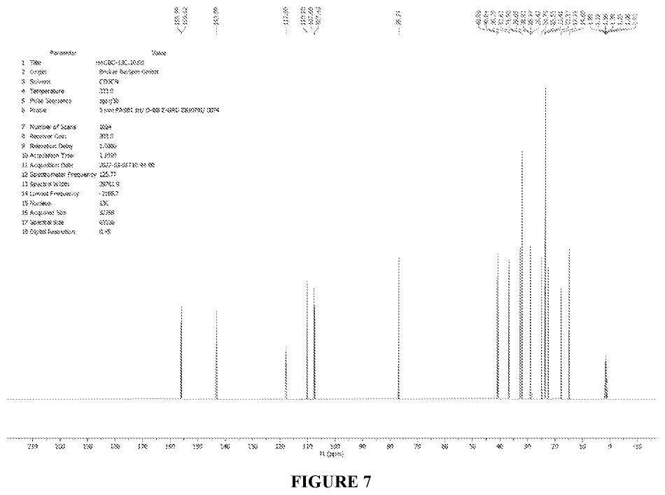

CBC was added to a polar protic or polar aprotic solvent in a reaction flask. A metal catalyst was added to the reaction flask under argon. A hydrogen source was added under argon slowly so as to avoid bumping the solution. The reaction mixture is stirred until complete by HPLC. H4CBC is an orange oil, FIG. 3B HPLC C₁₈: 9.891 min, FIG. 6 ¹H NMR (500 MHZ, MeCN) δ: 6.27 (2H), 2.70 (2H), 2.53 (2H), 1.84 (2H), 1.67 (5H), 1.53 (2H), 1.43 (4H), 1.32 (3H), 1.02 (9H). FIG. 7 ¹³C NMR.

### Example 4

### Synthesis of Hydrogenated CBC Using an Additive

To a two-neck round bottomed flask equipped with a reflux condenser was added CBC (2 grams), and the flask was charged with methanol (50-300 mL). The flask was purged of air using vacuum. The flask was then filled with argon. The purge/fill cycle was then repeated three times. Afterwards, ammonium formate (1 to 20 molar equivalents) was added to the round bottomed flask. Pd/C (0.1 to 5 molar equivalent by percentage of Palladium loading) was added to the reaction flask by way of a powder funnel. The reaction was then stirred until the reaction was observed to have been completed using HPLC as a guide. Once complete, the reaction mixture was then filtered over celite to remove the Pd/C. The mixture was then placed onto a roto evaporator to remove all methanol. The reaction mixture was then dissolved in hexane and was washed with water (10-100 mL, 3 times) in a separatory funnel. The aqueous layer was removed after each wash. The organic layer was then washed with a saturated brine solution (10-100 mL) and the aqueous layer was removed. The organic layer was then concentrated in vacuo. This orange oil was then be purified via distillation. Repeated chromatography can be employed as an alternative means for purification.

All of the methods disclosed and claimed herein can be made and executed without undue experimentation in light of the present disclosure. While the compositions and methods of this invention have been described in terms of preferred embodiments, it will be apparent to those of skill in the art that variations may be applied to the methods and in the steps or in the sequence of steps of the method described herein without departing from the concept, spirit and scope of the invention. More specifically, it will be apparent that certain agents which are both chemically and physiologically related may be substituted for the agents described herein while the same or similar results would be achieved. All such similar substitutes and modifications apparent to those skilled in the art are deemed to be within the spirit, scope and concept of the invention as defined by the appended claims.

What is claimed is:

1. A process for the preparation of a hydrogenated cannabigerol derivative, comprising:
providing a cannabigerol derivative of formula I to a reaction vessel; 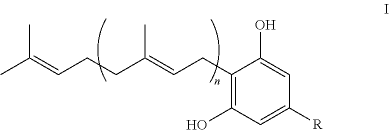
wherein n is an integer of from 0 to 4 and R is hydrogen or a substituted or unsubstituted alkyl, alkenyl, or aryl group having from 1 to 10 carbon atoms;
providing a catalyst to the reaction vessel;
providing hydrogen gas, a source of hydrogen gas, or a combination thereof to the reaction vessel; and
producing a hydrogenated cannabigerol derivative of formula II

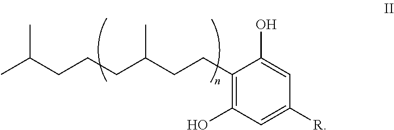

2. The process of claim 1, wherein R is methyl, ethyl, propyl, butyl, pentyl, hexyl, heptyl, dimethylheptyl, octyl, phenyl, or benzyl.

3. The process of claim 1, wherein the catalyst is provided in an amount ranging from 0.01 to 10 molar equivalents.

4. The process of claim 3, wherein the catalyst is selected from the group consisting of Pd/C, Pt/C, Rh/C, Ru/C, Raney nickel, Pd/alumina, Pd/activated charcoal, Pt/alumina, Pt/activated charcoal, Pt₂O (Adam's catalyst), Wilkinson's catalyst ([RhCl(PPh₃)₃]), Crabtree's catalyst ([CsH₁₂IrP(C₆Hₙ)₃CsHsN]PF₆), 9-borabicyclo[3.3.1]nonane, (R/S)-alpine borane, BH₃-DMSO, BH₃-THF, and N-methylimidodiacetic (MIDA) boronates.

5. The process of claim 4, wherein the catalyst is Pd/C or Pt/C.

6. The process of claim 1, wherein the hydrogen gas is provided in an amount that affords an intra-vessel gas pressure ranging from 1 bar to 20 bar.

7. The process of claim 1, wherein hydrogen gas is not provided to the reaction vessel.

8. The process of claim 1, wherein the source of hydrogen gas generates hydrogen gas in situ.

9. The process of claim 1, wherein the source of hydrogen gas comprises ammonium formate and/or formic acid.

10. The process of claim 9, wherein an amount of ammonium formate ranges from 1 to 40 molar equivalents.

11. The process of claim 9, wherein an amount of formic acid ranges from 1 to 40 molar equivalents.

12. The process of claim 1, further comprising providing a solvent to the reaction vessel.

13. The process of claim 12, wherein the solvent is selected from the group consisting of methanol, ethanol, propanol, isopropanol, butanol, tert-butanol, THF, 2-Me-THF, toluene, and ethyl acetate.

14. The process of claim 1, further comprising heating the reaction vessel.

15. The process of claim 14, wherein the reaction vessel is heated to a temperature ranging from 25° C. to 100° C.

16. The process of claim 1, wherein the reaction vessel is hermetically sealed.

17. The process of claim 1, further comprising purging the reaction vessel with an inert gas prior to addition of reactants and catalyst.

18. The process of claim 17, wherein the inert gas is nitrogen or argon.

19. A process for the preparation of a hydrogenated cannabichromene derivative, comprising:
providing a cannabichromene derivative of formula III to a reaction vessel; 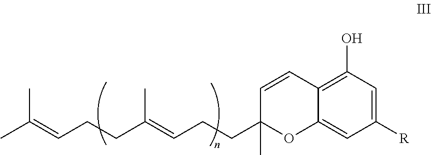
wherein n is an integer of from 0 to 4 and R is hydrogen or a substituted or unsubstituted alkyl, alkenyl, or aryl group having from 1 to 10 carbon atoms;
providing a catalyst to the reaction vessel;
providing hydrogen gas, a source of hydrogen gas, or a combination thereof to the reaction vessel; and
producing a hydrogenated cannabichromene derivative of formula IV

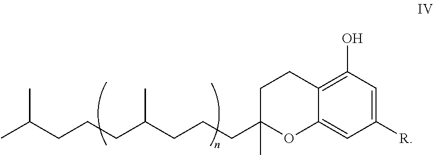

20. The process of claim 19, wherein R is methyl, ethyl, propyl, butyl, pentyl, hexyl, heptyl, dimethylheptyl, octyl, phenyl, or benzyl.

21. The process of claim 19, wherein the catalyst is provided in an amount ranging from 0.01 to 10 molar equivalents.

22. The process of claim 19, wherein the catalyst is selected from the group consisting of Pd/C, Pt/C, Rh/C, Ru/C, Raney nickel, Pd/alumina, Pd/activated charcoal, Pt/alumina, Pt/activated charcoal, Pt₂O (Adam's catalyst), Wilkinson's catalyst ([RhCl(PPh₃)₃]), Crabtree's catalyst ([CsH₁₂IrP(C₆Hₙ)₃CsHsN]PF₆), 9-borabicyclo[3.3.1]nonane, (R/S)-alpine borane, BH₃-DMSO, BH₃-THF, and N-methylimidodiacetic (MIDA) boronates.

23. The process of claim 22, wherein the catalyst is Pd/C or Pt/C.

24. The process of claim 19, wherein the hydrogen gas is provided in an amount that affords an intra-vessel gas pressure ranging from 1 bar to 20 bar.

25. The process of claim 19, wherein hydrogen gas is not provided to the reaction vessel.

26. The process of claim 19, wherein the source of hydrogen gas generates hydrogen gas in situ.

27. The process of claim 19, wherein the source of hydrogen gas comprises ammonium formate and/or formic acid.

28. The process of claim 27, wherein an amount of ammonium formate ranges from 1 to 40 molar equivalents.

29. The process of claim 27, wherein an amount of formic acid ranges from 1 to 40 molar equivalents.

30. The process of claim 19, further comprising providing a solvent to the reaction vessel.

31. The process of claim 19, wherein the solvent is selected from the group consisting of methanol, ethanol, propanol, isopropanol, butanol, tert-butanol, THF, 2-Me-THF, toluene, and ethyl acetate.

32. The process of claim 19, further comprising heating the reaction vessel.

33. The process of claim 19, wherein the reaction vessel is heated to a temperature ranging from 25° C. to 100° C.

34. The process of claim 19, wherein the reaction vessel is hermetically sealed.

35. The process of claim 19, further comprising purging the reaction vessel with an inert gas prior to addition of reactants and catalyst.

36. The process of claim 35, wherein the inert gas is nitrogen or argon.

37. A process for the preparation of tetrahydrocannabigerol (H4CBG) comprising:
providing cannabigerol (CBG) to a reaction vessel;
providing a catalyst to the reaction vessel;
providing hydrogen gas, a source of hydrogen gas, or a combination thereof to the reaction vessel; and
producing H4CBG.

38. A process for the preparation of tetrahydrocannabichromene (H4CBC), comprising:
providing cannabichromene (CBC) to a reaction vessel;
providing a catalyst to the reaction vessel;
providing hydrogen gas, a source of hydrogen gas, or a combination thereof to the reaction vessel; and
producing H4CBC.

39. A composition comprising a compound of formula II: 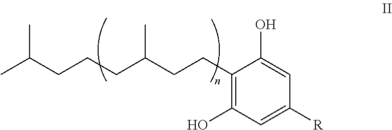
wherein n is an integer of from 0 to 4 and R is hydrogen or a substituted or unsubstituted alkyl, alkenyl, or aryl group having from 1 to 10 carbon atoms.

40. The compound of claim 39, wherein the compound is further defined as one of:

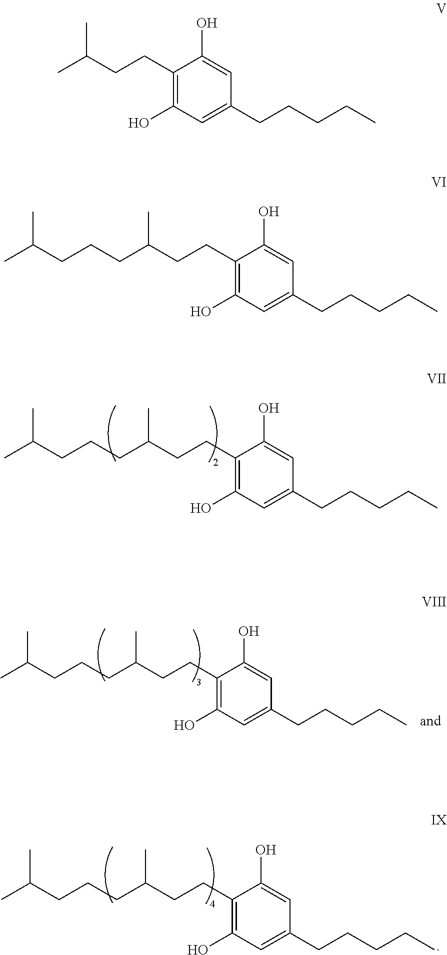

41. A composition comprising a compound of formula IV: 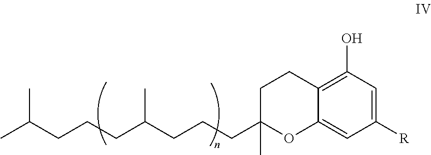
wherein n is an integer of from 0 to 4 and R is hydrogen or a substituted or unsubstituted alkyl, alkenyl, or aryl group having from 1 to 10 carbon atoms.

42. The compound of claim 41, wherein the compound is further defined as one of:

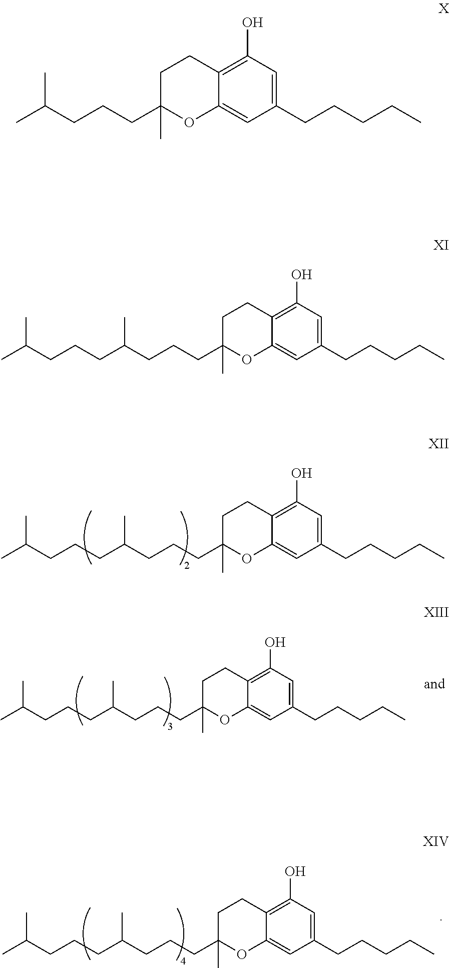

43. A pharmaceutical composition comprising a compound of any of claims 37 to 42.
